(19) United States
(12) Patent Application Publication
(10) Pub. No.: US 2011/0270320 A1
(43) Pub. Date: Nov. 3, 2011

(54) APPARATUS FOR BONE FIXATION

(75) Inventors: Jong-Keon Oh, Seoul (KR); Seo-Kon Kim, Gyeonggi-do (KR); Il Kim, Seoul (KR)

(73) Assignees: SOLCO BIOMEDICAL CO., LTD., Gyeonggi-do, KR (KR); Jong-Keon Oh, Seoul (KR)

(21) Appl. No.: 12/521,839

(22) PCT Filed: Dec. 31, 2008
(86) PCT No.: PCT/KR2008/007824

§ 371 Date: Jul. 21, 2011

(30) Foreign Application Priority Data
Nov. 21, 2008 (KR) 10-2008-0116188

(51) Int. Cl. A61B 17/80

(52) U.S. Cl. 606/290

(57) ABSTRACT

The present invention is characterized in that an apparatus for bone fixation that is composed of a plate and screws fixing the plate to a bone, includes: screws that has a head portion having a male thread on the outer circumference while increasing in diameter toward the upper portion in a tapered shape; washers of which the outer circumference is formed convex, and which has, on the inner circumference, a slope that contact with the head portion such that the head portion of the screw is placed and a female thread that is engaged with the male thread of the head portion; and a plate that has locking holes which are bored concave in the up-down direction and have a holding step, where the washer is placed, on the inner circumference, in which when the washer and the head of which the female thread and the male thread are partially engaged are inserted from above the locking hole and placed on the holding step, as the head portion is moved down by thread-fastening, the washer is in close contact and fixed in the locking hole while the diameter of the upper end increases, and the apparatus for bone fixation according to the present invention has the effect of inserting and fixing the screw perpendicular to the plate because the washer and screw are combined in advance and then fixed to the plate, and allowing an operator to more freely perform an operation because the operator can insert and fix the screw at a desired angle.

### CROSS-REFERENCE TO RELATED APPLICATION

This claims the benefit of priority to International Application No. PCT/KR2008/007824 having an International Filing Date of 31 Dec. 2008, which claims priority to Korean Application No. 10-2008-116188, filed on 21 Nov. 2008. The disclosure of these applications are hereby incorporated in their entirety by reference.

### BACKGROUND OF THE INVENTION

The present invention relates to an apparatus for bone fixation that joins broken bones, more particularly an apparatus for bone fixation designed to allow an operator to more freely perform an operation.

In general, for treating a bone fracture, apparatuses for bone fixation, which disposes and fix a plate to the surface of a broken bone with screws, for treatment, after the soft tissue of a bone fracture patient is cut, is used.

These apparatuses for bone fixation generally have structures in which holes are formed through a plate and screws are inserted in the holes.

However, there was a problem in that when the apparatuses for bone fixation in the related art are used over a long period of time after being transplanted into the patient's body, stress is concentrated around the screws due to external load, such that the screws loosen out of the bone and the plate. Reoperation was required, particularly for patients with osteoporosis.

### SUMMARY OF THE INVENTION

In order to overcome the problems, recently, studies about a locking system between the plate and the screw have been conducted. As an apparatus for bone fixation equipped with the locking system, a technology that prevents a screw from coming out by thread-fastening between the screw and a hole formed through a plate, by forming a female thread in the hole has been known in the related art.

### BRIEF DESCRIPTION OF THE DRAWINGS

FIG. 1 is an exploded perspective view of an apparatus for bone fixation according to a preferred embodiment of the present invention.

FIG. 2 is a cross-sectional view of a screw and a washer according to a preferred embodiment of the present invention.

FIG. 3 is a perspective view when the washer and the screw of the present invention are combined.

FIG. 4 is a perspective view when the washer of the present invention, combined with the screw, is fixed to a plate.

FIG. 5 is a flowchart illustrating that the apparatus for bone fixing of the present invention is fastened.

An embodiment of the present invention is described hereafter with reference to the accompanying drawings.

### DETAILED DESCRIPTION OF THE INVENTION

However, according to the locking system in the related art, it is impossible to adjust the insertion angle of the screw because the shape and insertion direction of the thread formed in the hole of the plate are fixed. Further, a drill guide that is threaded is connected to the inlet of the hole of the plate to insert the screw while maintaining a predetermined angle and the screw has to penetrate the bone, which needs to be transplanted, such that the work is complicated and time is unnecessarily wasted.

Further, after the bone fracture is repaired, the thread of the head portion of the screw sticks to the thread of the plate hole, such that it may be impossible to remove the screw. In this case, since it is required to cut the plate itself to be removed, several problems are generated, such as the cut area of the soft tissue increases, the operation time becomes long, and equipment for cutting the plate is needed. Furthermore, there is a risk in causing additional bone fractures while attempting to remove the plate by excessively lifting it.

In order to overcome the above problems, the present invention is characterized in that an apparatus for bone fixation that is composed of a plate and screws fixing the plate to a bone, includes: screws that has a head portion having a male thread on the outer circumference while increasing in diameter toward the upper portion in a tapered shape; washers of which the outer circumference is formed convex, and which has, on the inner circumference, a slope that contacts with the head portion such that the head portion of the screw is placed and a female thread that is engaged with the male thread of the head portion; and a plate that has locking holes which are bored concave in the up-down direction and have a holding step, where the washer is placed, on the inner circumference, in which when the washer and the head of which the female thread and the male thread are partially engaged are inserted from above the locking hole and placed on the holding step, as the head portion is moved down by thread-fastening, the washer is in close contact and fixed in the locking hole while the diameter of the upper end increases.

At least two or more gaps are formed at the upper end of the washer and the gaps are formed at a predetermined distance from each other.

The locking hole has a circular shape or a shape of which both sides are semicircular shapes facing each other at a predetermined distance from each other.

The upper end of the head portion is sloped to be in close contact with the slope of the washer when the washer is fixed in the locking hole while increasing in diameter.

The apparatus for bone fixation according to the present invention has the effect of inserting and fixing the screw perpendicular to the plate because the washer and screw are combined in advance and then fixed to the plate, and allowing an operator to more freely perform an operation because the operator can insert and fix the screw at a desired angle.

Since the washer of the present invention has the gaps, it can easily increase in diameter. A plurality of the gaps is formed at regular intervals and the upper end of the washer radially expands, such that it is stably fixed in the locking hole while preventing biasing to a side.

Further, the locking hole has a circular shape or a shape of which both sides are semicircular shapes facing each other at a predetermined distance, which allows an operator to be able to adjust the position and insertion angle of the screw in more detail, if necessary.

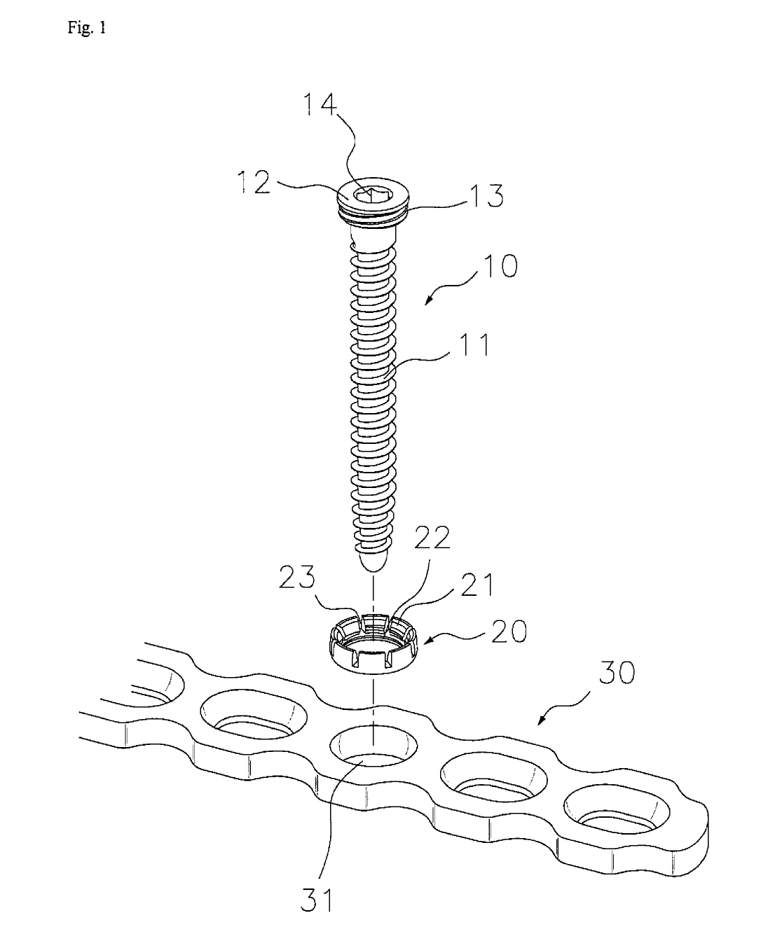

As shown in FIG. 1, the present invention is composed of screws 10, washers 20, and a plate 30.

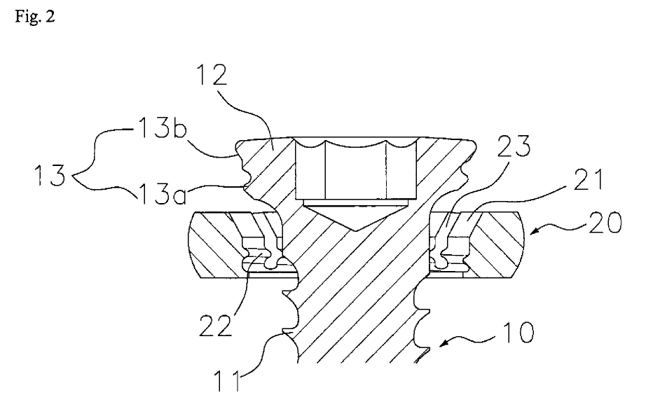

The screw 10 of the present invention has a threaded portion 11 where a thread is formed around the outer circumference to be inserted into a bone and a head portion 12 that is integrally formed at the upper end of the threaded portion 11. The screw 10 of the present invention, similar to the method of inserting a common screw, also has a wrench groove 14 on the upper surface of the head portion 12 to be inserted into a bone from above by a wrench. As shown in FIG. 2, the head portion 12 is tapered such that the diameter increases upward, and has a male thread 13a at the lower end of the outer circumference 13 and a slope 13b at the upper end.

The washer 20 of the present invention has a ring shape bored in the up-down direction at the center and a predetermined thickness, in which the outer circumference is formed convex, a female thread 22 that is engaged with the male thread 13a formed at the head portion 12 of the screw 10 is formed at the lower end of the inner circumference, and a slope 21 is formed at the upper end.

In addition, a plurality of gaps 23 is formed at regular intervals such that the upper end can radially open while the diameter of the upper end increases, when being pressed. It is preferable that the slope 13b of the head portion 12 and the slope 21 of the washer 20 have different angles such that the washer can be more easily opened, while the upper end of the washer 20 is opened by engagement of the male thread 13a with the female thread 22. That is, it is preferable to be configured such that the slope 13b of the head portion can be in close contact with the slope of the washer, only when the upper end of the washer 20 is the most open.

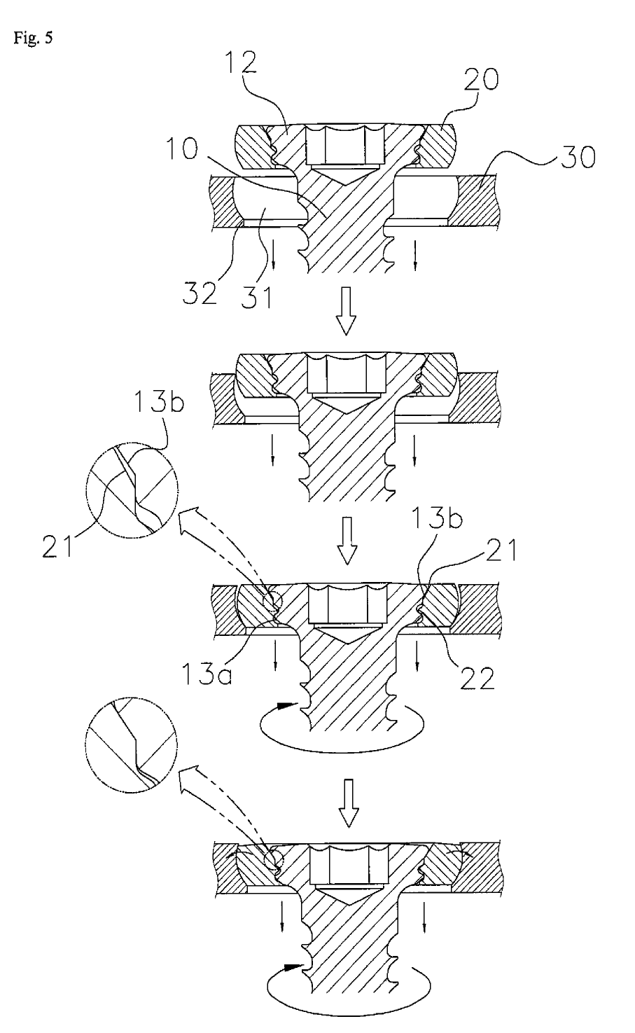

The plate 30 of the present invention has a plurality of locking holes 31. The locking hole 31 has a circular shape or a circular shape with both sides extending, that is, a shape of which both ends are semicircular shapes and spaced apart at a predetermined distance. As shown in FIG. 5, a holding step 32 is formed at the locking hole 31, such that the inner diameter of the lower end is smaller than the inner diameter of the upper end. Therefore, the washer 20 can be freely inserted and removed through the upper end of the locking hole 31, but cannot pass downward through the lower end.

Fastening of the present invention is described hereafter in detail with reference to the drawings.

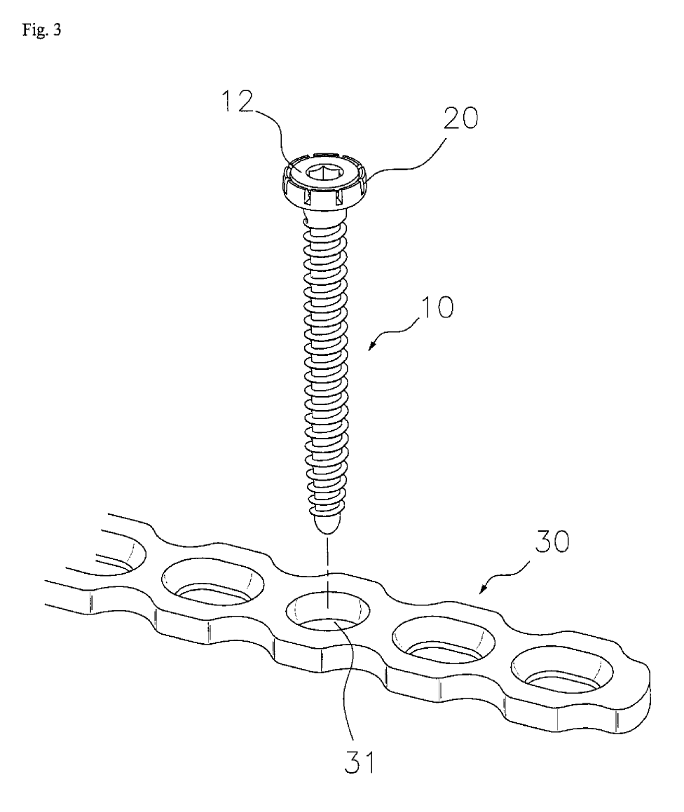

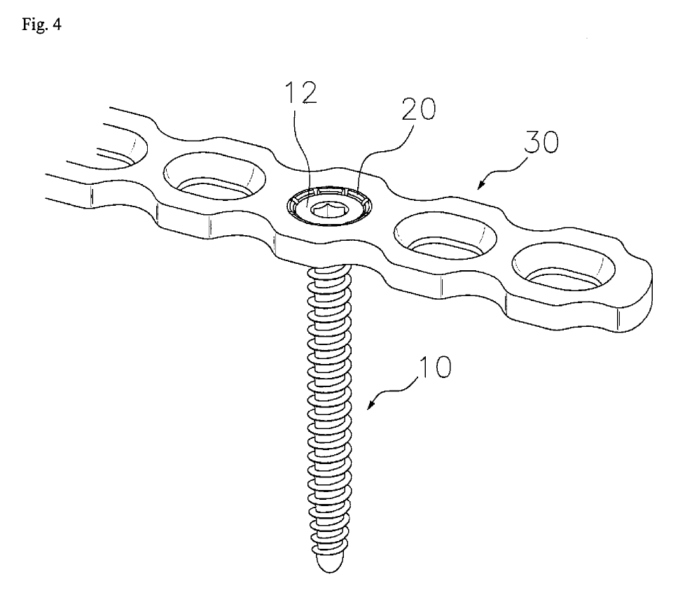

As shown in FIG. 3, the screw 10 of the present invention is combined with the washer 20 and inserted in the locking hole 31 of the plate 30, as shown in FIG. 4. That is, in an operation, the plate 30 is in contact with a bone and the screw 10 is inserted, while being combined with the washer 20, into the bone by a wrench (not shown).

The process of inserting the screw 10 is shown in FIG. 5. The washer 20 and screw 10, which are partially engaged in advance, are inserted into the locking hole 31 of the plate 30. As described above, the inner diameter of the upper end of the washer 20 is larger than the maximum outer diameter of the washer 20 such that the washer 20 can be freely inserted and removed, and the inner diameter of the lower end of the locking hole 31 is small such that the washer 20 cannot pass.

Therefore, as the screw 10 is inserted into a bone by a wrench, when the washer 20 combined with the screw 10 is placed on the holding step 32 of the locking hole 31, the washer 20 turned together with the screw 10 by the wrench is pressed at the lower end, such that it stops turning by frictional force.

Thereafter, the male thread 13a of the head portion 12, which continues being turned by the wrench, becomes engaged again with the female thread 22 of the washer 20, in which the head portion 12 is moved down by the engagement and the outer circumference 13 of the head portion 12 correspondingly presses the inner circumference of the washer 20 from the upper end. Accordingly, the washer 20 radially increases in diameter from the upper end, and as a result, it is in close contact and fixed to the inner circumference of the locking hole 31. In this position, as described above, since the slope 13b is formed at the upper end of the head portion 12, it is in close contact with the slope 21 of the washer.

This fastening method can be applied to when the locking hole 31 has, as described above, the shape of which both ends are formed in semicircular shapes and spaced apart at a predetermined distance from each other, as well as having circular shape.

The embodiments described herein and shown in the drawings just provide specific examples to help understanding the present invention, and do not limit the scope of the present invention. It should be understood by those skilled in the art that other modifications are possible on the basis of the spirit of the present invention, other than the embodiments described herein.

What we claim is:

1. An apparatus for bone fixation that is composed of a plate and screws fixing the plate to a bone, the apparatus comprising:
screws that have a head portion having a male thread on the outer circumference while increasing in diameter toward the upper portion;
washers of which the outer circumference is formed convex, and which has, on the inner circumference, a slope that contacts with the head portion such that the head portion of the screw is placed and a female thread that is engaged with the male thread of the head portion; and
a plate that has locking holes which are bored concave in the up-down direction and have a holding step, where the washer is placed, on the inner circumference,
wherein when the washer and the head of which the female thread and the male thread are partially engaged are inserted from above the locking hole and placed on the holding step, as the head portion is moved down by thread-fastening, the washer is in close contact and fixed in the locking hole while the diameter of the upper end increases.

2. The apparatus for bone fixation according to claim 1, wherein gaps are formed at the upper end of the washer.

3. The apparatus for bone fixation according to claim 2, wherein at least two or more of the gaps are formed at regular intervals.

4. The apparatus for bone fixation according to any one of claims 1 to 3, wherein the locking hole is formed in a circular shape.

5. The apparatus for bone fixation according to any one of claims 1 to 3, wherein the locking hole is formed in a shape of which both ends are semicircular shapes facing each other and extended at a predetermined distance from each other.

6. The apparatus for bone fixation according to any one of claims 1 to 3, wherein the upper end of the head portion is sloped to be in close contact with the slope of the washer when the washer increases in diameter and is fixed in the locking hole.
